(19) United States
(12) Patent Application Publication
(10) Pub. No.: US 2025/0332334 A1
(43) Pub. Date: Oct. 30, 2025

(54) CANISTER FOR AUTOLOGOUS FAT TRANSFER

(71) Applicant: Wells Johnson Company, Tucson, AZ (US)

(72) Inventors: John Francis Wells, Tucson, AZ (US); McKay S. Crowder, Tucson, AZ (US); Yakov S. Sidorin, Tucson, AZ (US)

(21) Appl. No.: 19/175,836

(22) Filed: Apr. 10, 2025

Related U.S. Application Data
(60) Provisional application No. 63/640,570, filed on Apr. 30, 2024.

(51) Int. Cl. A61M 1/00

(52) U.S. Cl. CPC A61M 1/893

(57) ABSTRACT

A gadget for use in harvesting and transfer of fat tissue, including a canister dimensioned to operate in absence of stirring (whether manual or mechanical) and filtering of lipoaspitate and to remove the unwanted fluid portion of lipoaspirate without the use of vacuum.

### CROSS-REFERENCE TO RELATED APPLICATIONS

This US patent application claims priority from and benefit of the U.S. provisional patent application No. 63/640,570 filed on Apr. 30, 2024, the disclosure of which is incorporated by reference herein.

### TECHNICAL FIELD

This invention generally relates to surgical equipment and, in particular, to a canister configured for use in harvesting and transfer of fat tissue such as autologous fat transfer.

### RELATED ART

In aesthetic body contouring, it is sometimes desirable to transfer fat tissue from one portion of the body (where the presence of the fat makes the body appear excessive or unsightly, for example) to another portion of the body to re-shape such other portion of the body to what is considered to be more appropriate or desired. One typical well-known situation is the removal of fat from a person's abdominal, hip, or thigh region and transfer of this fat to the buttocks or breast region. Such procedure is referred to in related art as autologous fat transfer (or AFT).

The most common approach to fat tissue removal is liposuction, which first involves the infiltration of fluid into a selected region of the body, optionally followed by the application of ultrasound to fragment or emulsify the fat tissue to separate it from the connective tissue matrix and completed by removal of the fat/fluid mixture with the use of vacuum (or, alternatively, mechanically activated) cannula.

When the fat tissue is removed by liposuction, such removed fat tissue is in a form of a lipoaspirate that includes fat and well as remnant infiltration fluid, blood and other unwanted connective tissues (the latter collectively referred to herein as unwanted fluids). Accordingly, the removed from the body fat tissue is in the form rather unsuitable for reinjection into the patient. In order to separate the wanted fat component from the overall lipoaspirate fluid before the fat component is reinjected into a patient, a separation device is employed.

A skilled person will readily appreciate that to the extent possible, it is desirable to have the processes of harvesting, separating, and reinjecting take place under conditions approaching aseptic, in order to prevent introduction of unwanted infectious or otherwise harmful material into the patient. Improvement of the separation step of the overall AFT process remains of interest in related art, as the long-term success of reinjection depends on no small part on the material quality of the separated fat.

A number of approaches have been attempted to solve the separation process issues. In the simplest case, the lipoaspirate is allowed to sit in a harvest container for a period of time. The fat component of the lipoaspirate, being less dense than either blood, freed oils, or saline, eventually floats to the top region of the container where it can be decanted.

The use of centrifuging the lipoaspirate is sometimes employed to separate the fat cells from the other fluids.

To minimize or even completely avoid manual handling and manipulation of the lipoaspirate (which procedure understandably increases the chance of biological contamination of the lipoaspirate) a specifically-structured container may be used that includes a removal port at the bottom of the container. Such removal port eliminates the need to decant the fat. Instead, after the necessary settling time, the appropriate valve cooperated with the removal port can be opened and the (heavier than fat itself) blood/fluid mixture can be drawn off until only the fat remains in the container. (The port then may be connected to an appropriate injection syringe for fat transfer for reinjection.) An example of such container is provided by the “Origins Lipo Harvesting System” canister (Sound Surgical Technologies, Louisville, CO).

Various other approaches—that are referred to, for example, in U.S. Pat. No. 8,858,518 (the entire disclosure of which is incorporated by reference herein)—have been attempted, which require (i) the use of an internal (that is, inside the container, and typically removable) filter and/or a screen with openings/filtering characteristics that have to be judiciously chosen for passing a desired amount of fat therethrough, and/or (ii) a vacuum port below such filter/screen to draw the fluid from below such filter/screen and even (iii) a vane allowing for manual agitation of the fat within the canister while under aspiration vacuum. The related art does not seem to provide any specific discussion or observation as to whether such structural additions to the canister really improve the purity of fat separated for transfer and reinjection-they may or may not. At the same time, such additions and modifications definitely complicate the structure of the canister and require attention of the clinician to the operation of the canister (in other words, the procedure of fat separation no longer remains passive). One practical shortcoming of the structures used by related art has been, however, practically proven repeatedly: the substantially flat bottom of a canister of related art does not facilitate—and, in most of the cases, complicates—the collection of all the material substance from the canister via the outlet port.

Furthermore, the substantially optically opaque or at least optically translucent nature of a body of a canister of related art—and, more specifically, the lack of visibility of what is happening inside the outlet port and/or a connector leading from the volume of the canister to the outlet port and/or in and immediately under the aspiration port (typically constructed at or on the lid of the canister) presents a substantial practical complications in operation of the conventional canister. Specifically, a typically multi-use canister simply cannot and should not be re-used if there remains any residue of the biological matter from the previous use, in order to not jeopardize the health of the next patient.

### SUMMARY OF THE INVENTION

Embodiments of the invention provide an article of manufacture that includes a canister that has a body and a first axis and that is configured for use in autologous fat transfer and having a first axis. The body includes a side wall, a bottom, an outlet port attached to the body, and a tubular element. The bottom has an inner surface inclined with respect to the first axis and a drain formed in such inner surface. (Optionally, and substantially in every implementation of the article of manufacture, the inner surface may be dimensioned as a substantially conical surface. When this is, indeed, the case, such inner surface may be substantially centered at the first axis and/or an outer surface of the side wall may be dimensioned as a yet another substantially conical surface.) The side wall and the bottom limit a volume of the canister, which volume includes a lower volume portion and an upper volume portion. The lower volume portion is limited by the inner surface and a plane that is substantially normal to the first axis and that passes through an upper-most point of the inner surface, while the upper volume portion is limited by such plane and an upper edge of the side wall. The tubular element has a second axis and is configured to fluidly connect the train and the outlet port. (Optionally, the tubular element may be reversibly removed from the body of the canister and/or the second axis may be tilted with respect to the first axis. Optionally, a third axis of the outlet port may be inclined with respect to the first axis and/or be substantially parallel to the second axis.) The article is devoid (that is, does not contain) a component or device structured to stir contents of the canister. Alternatively or in addition, the article may be structured to satisfy at least one of the following conditions: to include an aspiration port in fluid communication with the upper volume, and to not contain (that is, to be devoid of) a filter configured to filter out a portion of the contents of the canister upon removal thereof through the outlet port. Alternatively or in addition—and substantially in every implementation of the article—the body may additionally include a base that is circumferential to the first axis and that engages the side wall below the bottom, while the outlet port is attached to the base optionally at a level that is farther away from the upper edge than a level of the drain (in which case the axis of the tubular element and the first axis are inclined or tilted—not parallel or perpendicular—with respect to one another). In at least one specific case, the base may include an upper base portion merging with the side wall and a pedestal portion that merges with the upper base portion and that has a diameter exceeding a diameter of the upper base portion, and/or a thickness of a wall of the base and a thickness of the side wall may be substantially equal to one another and remain substantially the same at any location at the article of manufacture. Optionally, an upper surface of the pedestal is dimensioned substantially as an annulus and/or the upper surface of the pedestal is not located in a plane.

Embodiments of the invention additionally provide an article of manufacture that includes a canister that is configured for use in autologous fat transfer and that has a body with a first axis. The body includes a side wall; a bottom having an inner surface inclined with respect to the first axis and a base. (The side wall and the bottom limit a volume of the canister). The body further includes a top removably engaged with the side wall to create a fluid seal with the side wall; an outlet portion attached directly to the based on fluid communication with the volume of the canister; and a tubular element configured below the bottom to fluidly connect the inner surface and the outlet port (such tubular element has a second axis that is tilted or inclined—not parallel or perpendicular—with respect to the first axis. The base has a base wall with a base height, the base wall is circumferential to the first axis and engages (whether seamlessly or with a spatial kink or spatial depression) the side wall below the bottom. Optionally, the first and second axes may intersect. In at least one specific case, a lower edge of the base wall may be dimensioned to define a base aperture leading to a base volume (which based volume is limited by an outer surface of the bottom and the base wall) while the tubular element may be substantially completely disposed within such base volume. Alternatively or in addition, each portion of substantially every implementation of the article may be configured to be optically transparent.

Embodiments further provide a method that includes performing the following steps using substantially every embodiment of the article of manufacture: a step of maintaining a lipoaspirate matter contained within the volume of the canister without agitation to have such lipoaspirate matter separate into an unwanted fluid component in contact with the inner surface of the bottom and a fat component on top of the unwanted fluid component; and a step of extracting the unwanted fluid component through the outlet port along the second axis.

### BRIEF DESCRIPTION OF THE DRAWINGS

The invention will be more fully understood by referring to the following Detailed Description of Specific Embodiments in conjunction with the Drawings, of which:

FIGS. 1, 2, 3, 4, 5, 6, 7, and 8 provide, respectively, a first perspective view, a front elevational view, a rear elevational view, a first side elevational view, a second side elevational view, a top plan view, a bottom plan view, and a cross-section view of an embodiment of the canister structured according to the idea of the invention. The base of the canister (which includes a pedestal) is substantially seamlessly merging with a side wall thereof. The base is dimensioned to have the outlet port protrude outwards therefrom.

FIGS. 9, 10, 11, 12, 13, 14, 15, and 16 provide, respectively, a first perspective view, a front elevational view, a rear elevational view, a first side elevational view, a second side elevational view, a top plan view, a bottom plan view, and a cross-section view of a related embodiment of the canister structured according to the idea of the invention. The base of the canister (which includes a pedestal) is substantially seamlessly merging with a side wall thereof. The base is dimensioned to the outlet port be harbored or lodged in a notch or opening formed in the base portion and forming the outlet port receptacle.

FIGS. 17A, 17B schematically illustrate a lower portion of a related embodiment of the canister structured according to the idea of the invention. Here, the base of the canister includes a pedestal and an upper base portion dimensioned to have a smaller diameter than those of the body of the canister and the pedestal, thereby forming a structural “neck”.

FIGS. 18 and 19 present yet two more views of an embodiment of the invention—this time, the canister is shown complemented with a lid (top portion) that is equipped with latches and an appropriate O-ring to sealingly secure the lid onto of the canister, the lid carrying an aspiration port and a lid handle. Latches and/or the O-ring are optionally structured to be separable from the lid.

Generally, the sizes and relative scales of elements in Drawings may be set to be different from actual ones to appropriately facilitate simplicity, clarity, and understanding of the Drawings. For the same reason, not all elements present in one Drawing may necessarily be shown in another. While specific embodiments are depicted in the figures with the understanding that the disclosure is intended to be illustrative, these specific embodiments are not intended to limit the scope of invention the implementations of which are described and illustrated herein.

### DETAILED DESCRIPTION

Various embodiments are described more fully below with reference to the accompanying drawings, which form a part hereof, and which show specific exemplary embodiments for practicing the invention. However, embodiments may be implemented in many different forms and should not be construed as limited to the embodiments set forth herein; rather, these embodiments are provided so that this disclosure will be thorough and complete, and will fully convey the scope of the invention to those skilled in the art. Embodiments may be practiced as methods, systems or devices. The following detailed description is, therefore, not to be taken as limiting.

According to the idea of the present invention, embodiments of the canister for autologous fat transfer are judiciously dimensioned to maintain the process of separation of fat (from the collected lipoaspirate) substantially passively while, at the same time, ensuring that substantially all unwanted material matter is extracted from the canister through a corresponding outlet port during the conventional extraction process, whether with or without the use of a vacuum pump. (In addition, at least in some cases, the implementations of the idea of the invention allow the user to avoid the need in the use of filters and/or screens and the need in extraction of either such unwanted material matter (that is, remnant infiltration fluid, blood, and other unwanted connective tissues, interchangeably referred to herein as “unwanted fluid”) or the separated fat from the canister.) To achieve this goal, both the internal surface of the bottom of a body of the canister and the tube, connecting the drain formed in the bottom with the outlet port of the canister, are necessarily inclined with respect to a plane that is perpendicular to the axis of the body of the canister-thereby, in stark contradistinction with designs of the related art, providing the canister not only with a specific/dedicated portion of the volume in which the unwanted fluid is naturally congregated under gravity but also with the substantially gravity-driven flow of the contents of the container through the output port.

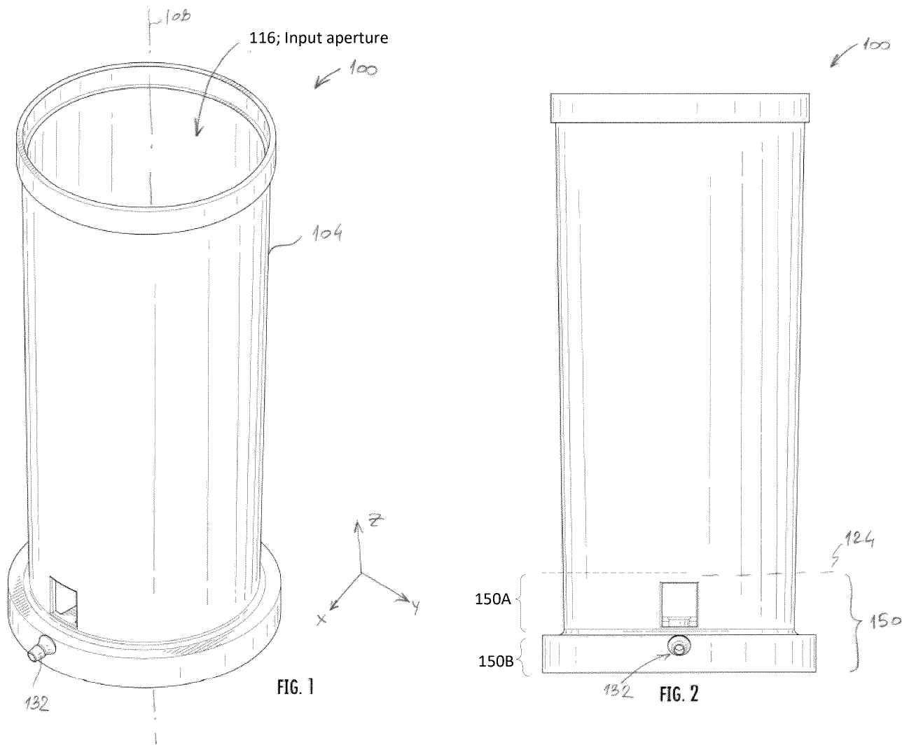

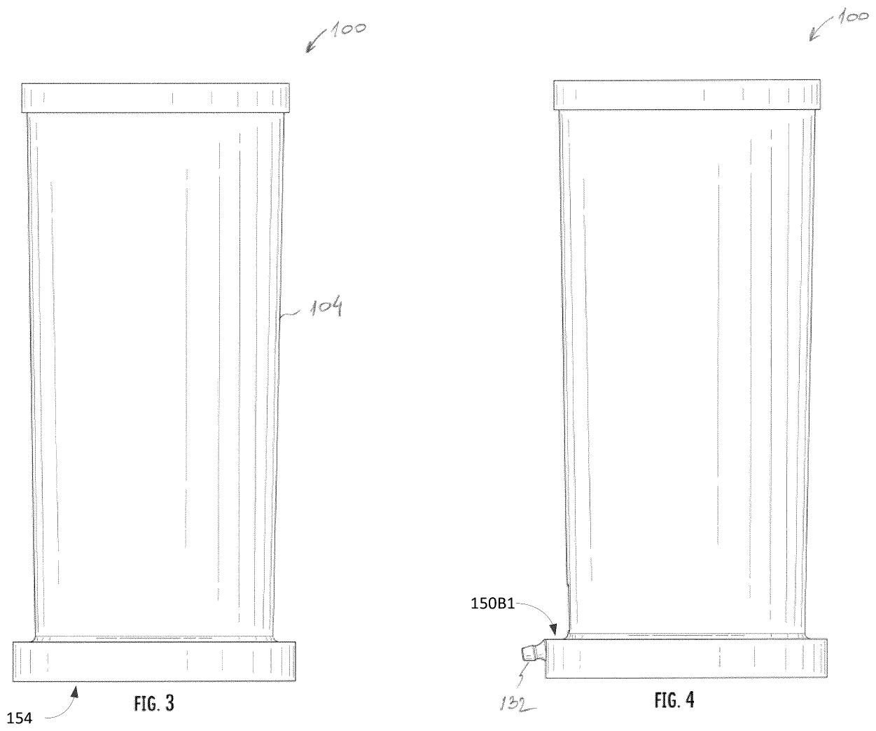

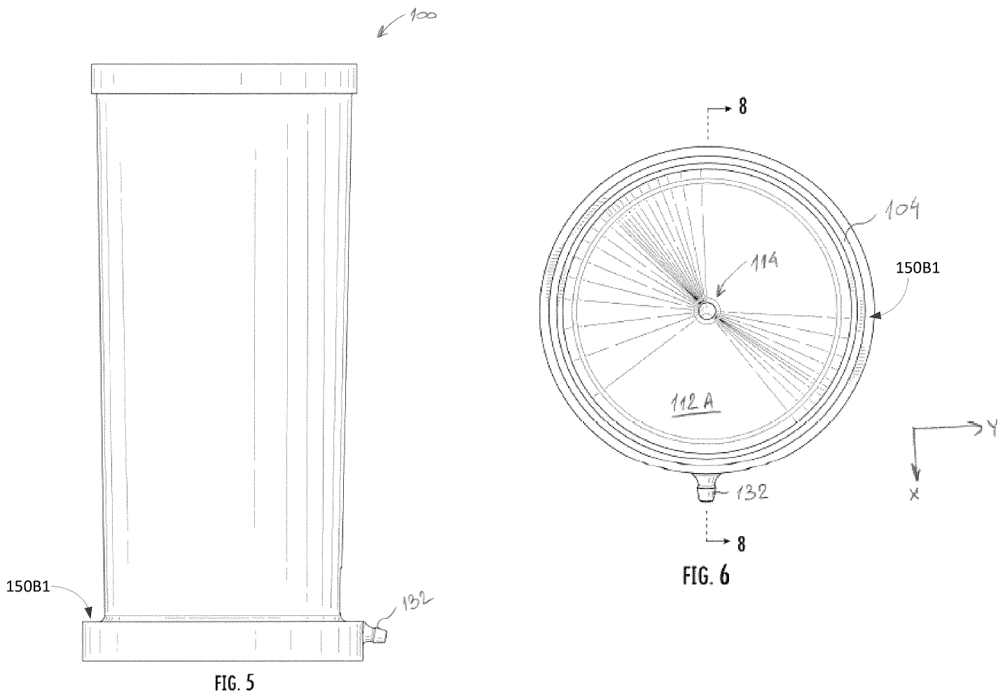

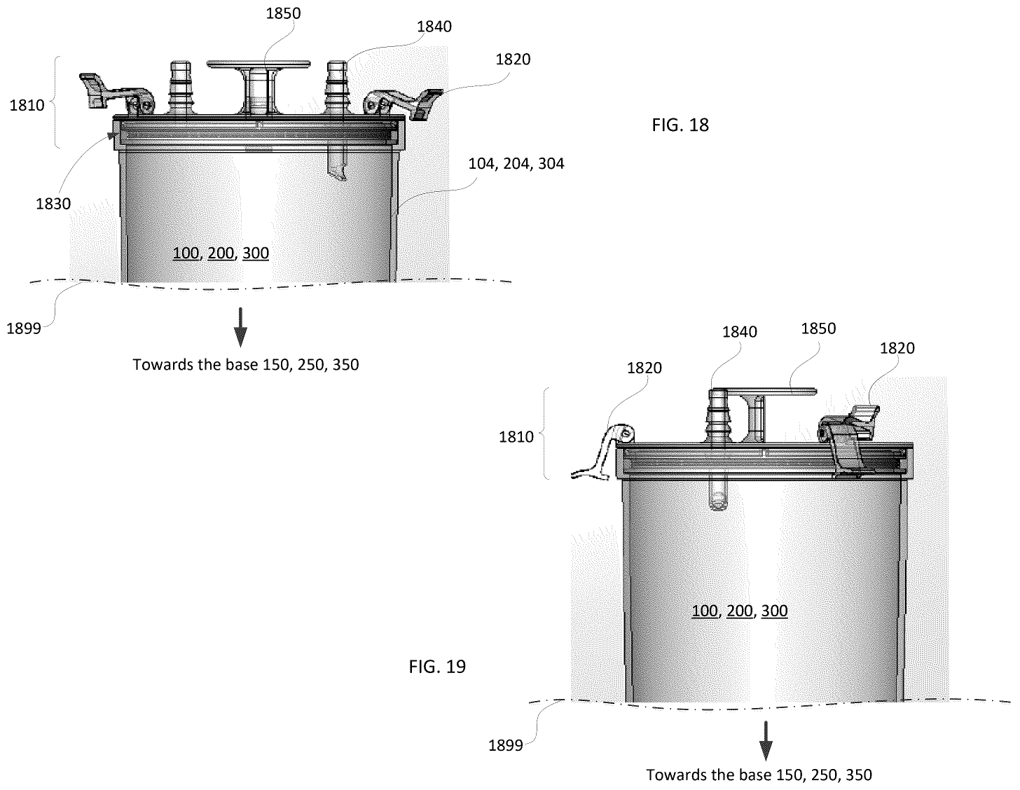

One embodiment is schematically illustrated in FIGS. 1, 2, 3, 4, 5, 6, 7, and 8. Here, FIGS. 1 and 8 depict (in a perspective view and in a cross-sectional view, respectively) a canister 100 of an article of manufacture having an axis 108 as well as the body with a side wall 104 (which, in one specific case, is substantially cylindrical and in a related case—substantially conical such as to be tilted with respect to the axis 108) and the bottom 112. The upper (internal to the body) surface 112A of the bottom 112 is generally inclined with respect to the axis 108 (as shown in FIGS. 6 and 8, for example). The bottom 112 contains a drain opening 114 therethrough, see, e.g., FIGS. 6, 8 (As shown in the specific example of FIG. 8—the upper surface 112A is a substantially conical surface.) The canister 100 is shown without a top portion or lid, which is generally optional but preferred, and which—when sealingly engaged with the side wall 104—may carry an appropriate aspiration port that is in fluid communication with the volume 116 of the canister (as discussed below in reference to FIGS. 18, 19). Access to the volume 116 is otherwise provided through the input aperture of the body of the canister.

The volume 116 of the body of the embodiment 100 can be subdivided into a lower volume 120 and an upper volume 128. The lower volume 120 is limited by a plane 124 (that is substantially normal to the axis 108 and that passes substantially through an upper-most point of the inner surface 112A) and the inner surface 112A itself. The upper volume 128 is limited by the plane 124 in the lower part of the canister and an upper edge of the side wall 104.

The opening of the drain 114 (formed throughout the bottom 112) is fluidly connected with the outlet port 132 via a tubular body or element 136 (having an axis 140 that is tilted with respect to the axis 108 to maintain the outlet portion 132 in substantially passive fluid communication with the volume 116—that is, such fluid communication that generally does not require any input applied to a fluid contained within the lower volume to propagate towards and through the outlet port 132). The axis 144 of the outlet port 132 may optionally be also tilted with respect to the axis 108 (forming an angle of about 9-10 degrees with a horizon, in one non-limiting example) and, in a specific case, be optionally substantially co-incident with the axis 140 of the tubular body or element 136. (In another implementation, the axis 144 of the outlet port 132 may be tilted with respect to the axis 140 of the tubular element 136.)

In at least the specific case illustrated by FIGS. 1 through 8, the embodiment 100 also includes a base portion 150 (a base, for short) that extends below the plane 124 and below the bottom 112, that is substantially circumferential with respect to the axis 108, and that is substantially seamlessly merging with the side wall 104. As shown, the base includes an upper base portion 150A and a pedestal 150B, the outer diameter of which is larger than that of the upper base portion 150A. The pedestal 150B has an upper surface 150B1 (clearly seen in FIG. 6, for example, which is dimensioned substantially as an annulus.

As the skilled artisan will readily appreciate, at least in one particular case when the embodiment 100 is manufactured via molding, the surface 150B1 is intentionally dimensioned to deviate from a planar surface to make the molding process repeatable and simplified. For the same reason, when the embodiment 100 is manufactured via molding, thicknesses of the side wall 104, a wall of the upper base portion 150A, and a wall of the pedestal 150B are made substantially equal to one another and, in addition and preferably—substantially unchanged across such walls to ensure the substantially spatially uniform and un-agitated flow of the molten material in the course of manufacturing. Furthermore, the judicious choice of an even, unchanged wall thickness of the overall embodiments causes the process of cooling of the just-molded embodiment to be spatially balanced, which in turn increases the material strength and/or removes the internal stresses.

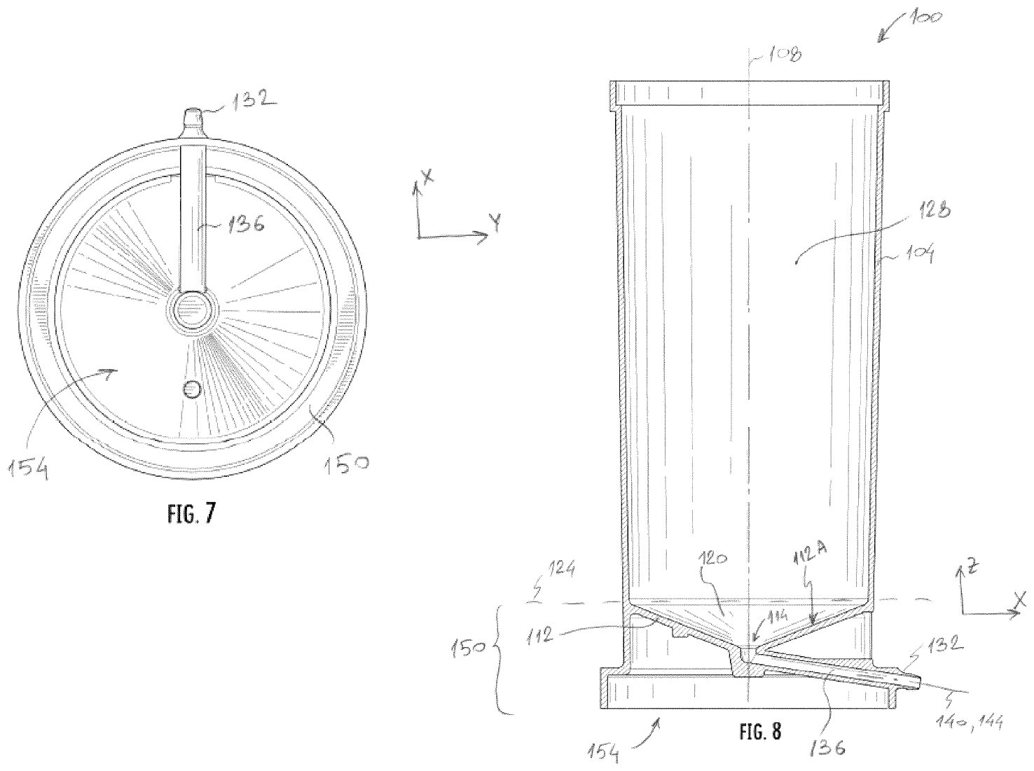

The wall of the base 150 defines an aperture 154—in the pedestal 150B of the embodiment 100—that leads from to a base volume that is limited by the wall of the base and the lower surface of the bottom 112. It is in this base volume that the tubular element 136 is at least partially located, see FIGS. 7, 8. While in at least one specific case the tubular element or body 136 may be pre-molded (and thus be formed at least in part substantially monolithic with the bottom 112 and/or the base 150 and/or the outlet port 132, as shown in FIG. 8), optionally such tubular element may be configured to be separable from the rest of the embodiment—especially in the case when the outlet port 132 is configured as a reversibly removable from the base 150 component of the embodiment.

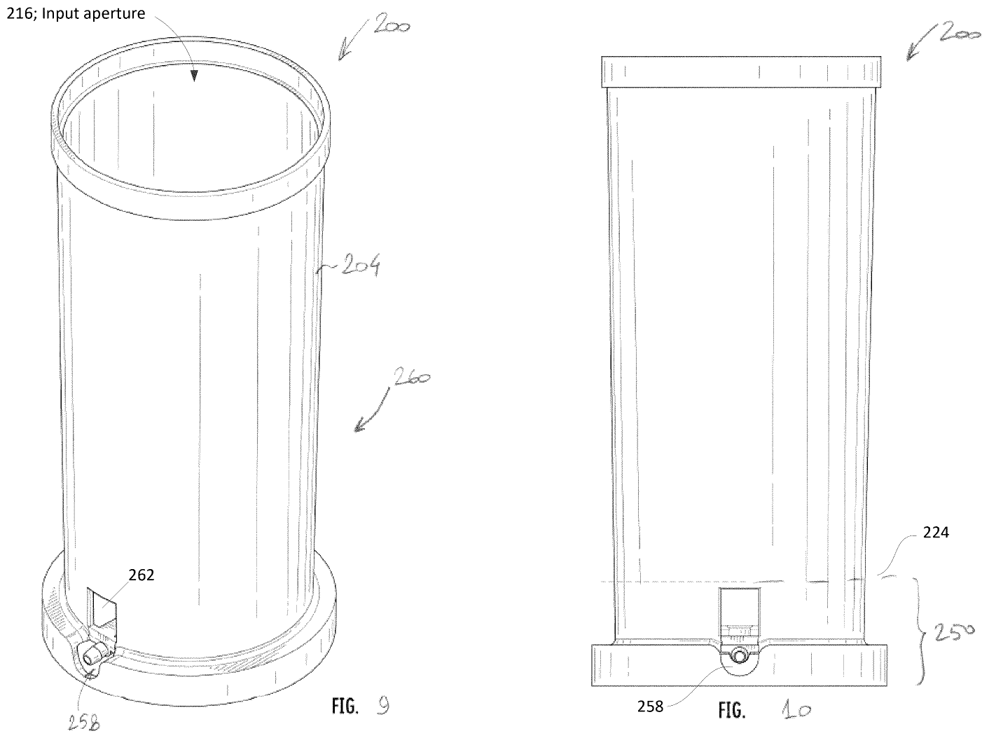

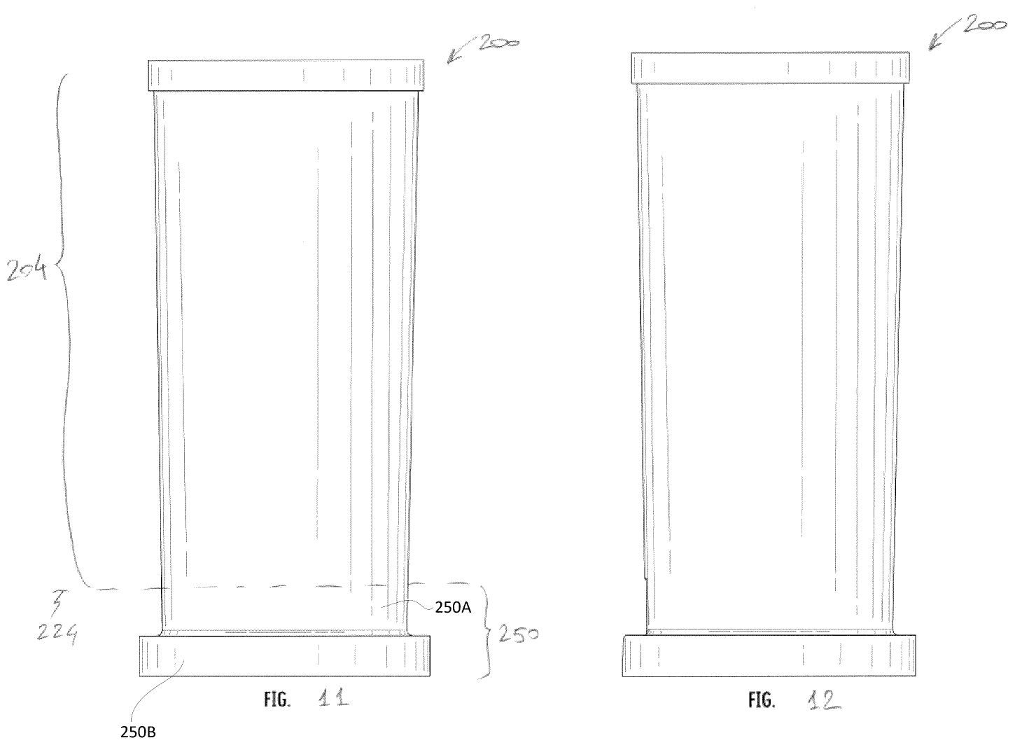

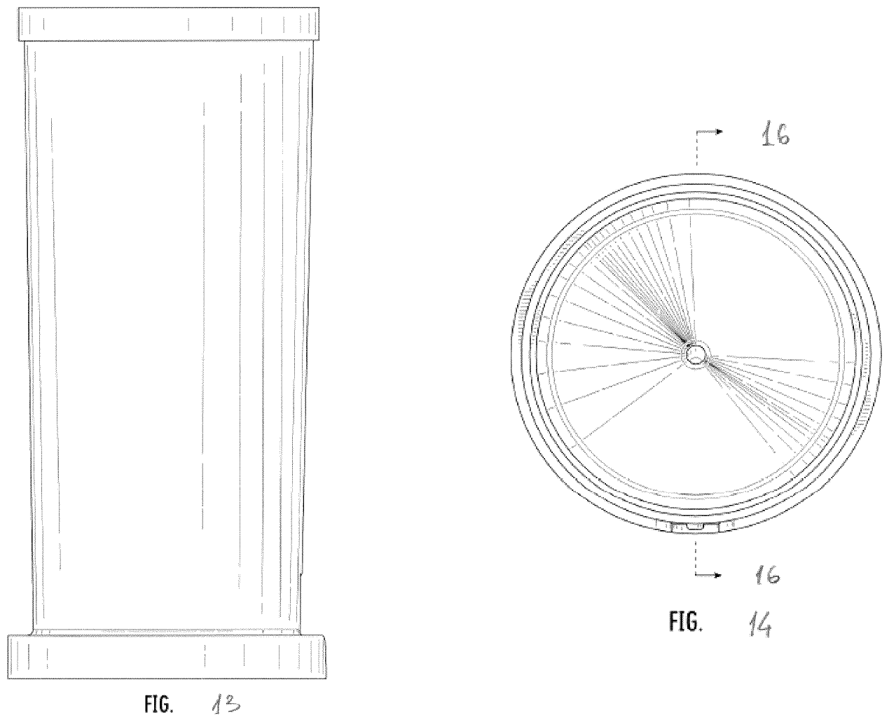

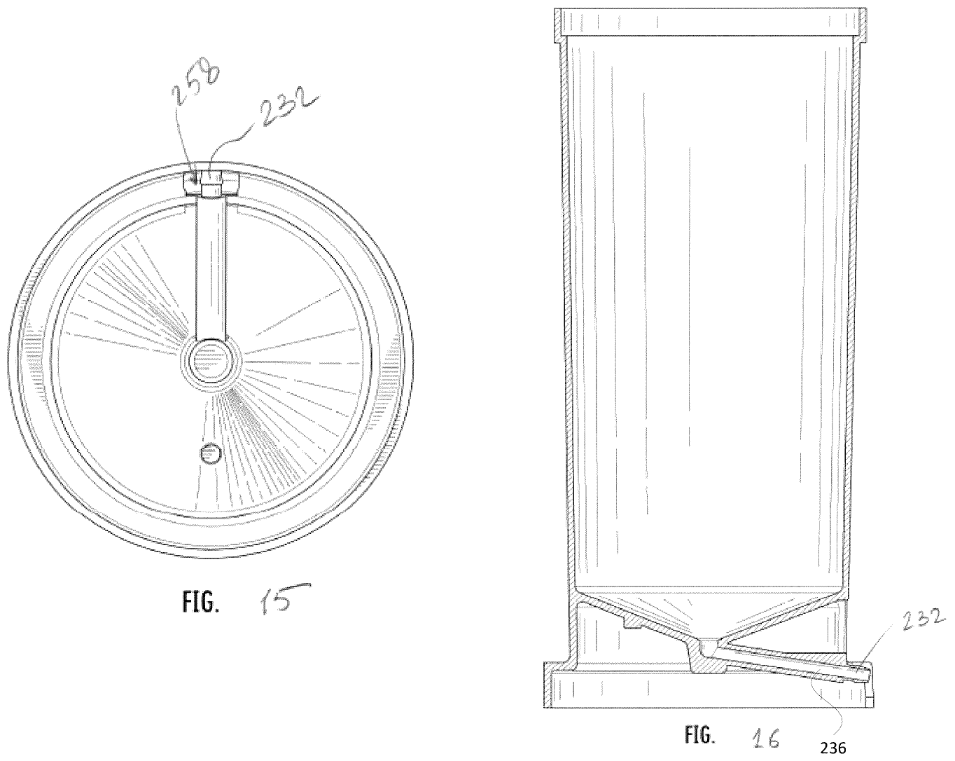

As shown in the embodiment 100 of FIGS. 1 through 8, the outlet port 132 is dimensioned to protrude outside the side wall 104 and/or the wall of the base 150 (as shown-protrude outside of the bounds of the pedestal 150B). This, in practice, may under some circumstances limit the operability of the canister 100 in that the outlet port 132 can be accidentally “knocked off” the canister, thereby rendering the canister substantially inoperable for the intended purpose of autologous fat transfer. Such practical problem is addressed by a related embodiment 200, shown in corresponding FIGS. 9, 10, 11, 12, 13, 14, 15, and 16, the structure of which is substantially similar to that of the embodiment 100. (The local system of coordinates for FIGS. 9-16 is substantially the same as that for FIGS. 1-8.) Access to the volume 216 of the canister is provided through the input aperture of the body of the canister. Here, however, the base 250 (extending below the plane 224 that is defined in a fashion analogous to that of plane 124 of FIG. 8 and including the upper portion 250A of the base and the pedestal 250B) contains an opening or a notch 258 dimensioned such as to substantially completely accommodate/harbor/lodge the outlet port 232 therein. The outer surface 260 of the overall canister is now defined by the outer surface of the side wall 204 and the outer surface of the base 250.

In some specific cases, when the embodiment of the canister is fabricated with the use of the molding process, the proper and optimized completion of the manufacture may require a formation of a “window”—shown as 262 in the base 250 of the embodiment 200—dimensioned to fit the tools used during the molding process. Whether present or not, such a window does not affect the mechanical and/or operational properties of the resulting embodiment.

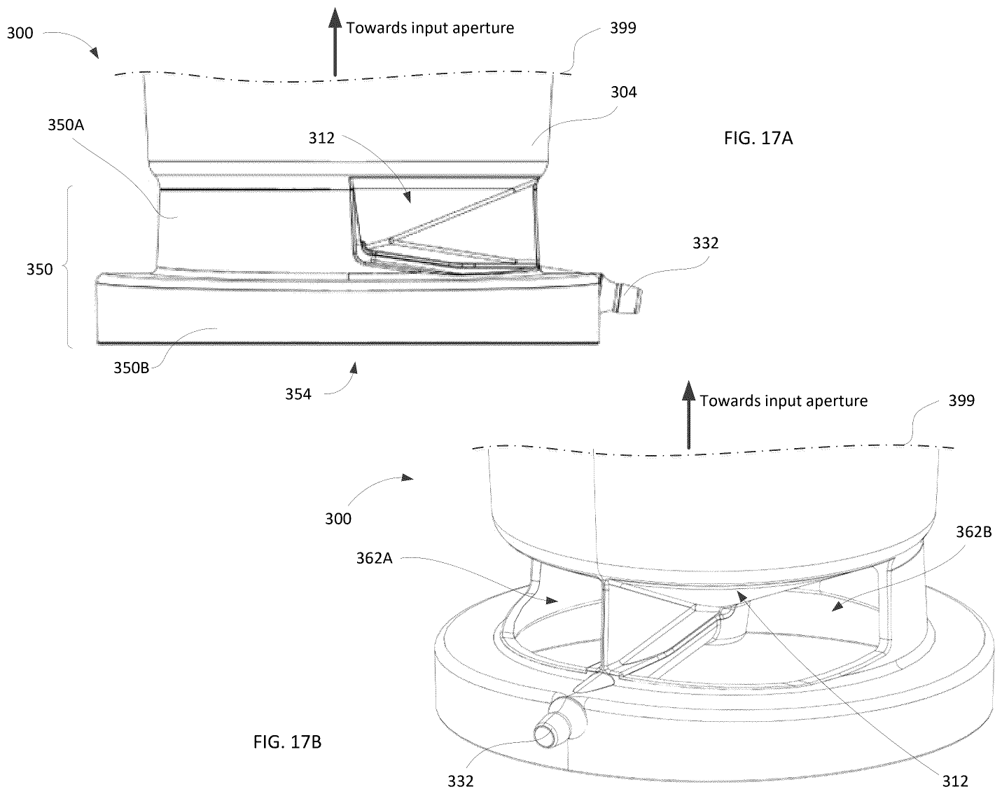

To this end, FIGS. 17A, 17B schematically illustrate-in side and isometric views, respectively-a lower portion of a related embodiment 300. (The dash-dot line 399 is a break line indicating the not-shown portion of the embodiment.) Here, the base 350 is dimensioned to also include an upper portion 350A and a pedestal 350B, while the upper portion 350A is dimensioned to be substantially smaller in diameter as compared with both the pedestal 350B and the side wall 304 of the body of the embodiment 300, thereby forming a “neck” of the embodiment 300. The design of the molding process for fabricating this embodiment requires the presence of two fabrication-assisting openings 362A, 362B in the base 350, providing additional access to the base volume (limited by the wall of the base and the lower surface of the bottom 312, partially visible through the openings 362A, 362B). Additional access to the base volume is provided by the aperture 354 defined in the pedestal 350B (by analogy to that discussed in relation to embodiment 100).

FIGS. 18 and 19 schematically illustrate the same portion of an embodiment of the article of manufacture structured according to the idea of the invention, at different angles and in different operational states. (The dash-dot line 1899 is a break line indicating the not-shown portion of the embodiment.) Here, the canister (such as 100, 200, 300) is complemented with a lid (top portion) 1810 that is equipped with latches 1820 and an appropriate O-ring 1830 to sealingly secure the lid 1810 on top of and within the bounds of the sidewall of the canister (the lid 1810 can be seen to carry an aspiration port 1840 and a lid handle 1850). Latches and/or the O-ring may optionally be structured to be separable from the lid

As the skilled person will now readily appreciate, in stark contradistinction with embodiments of the related art, an article of manufacture containing any and every of the embodiments 100, 200, 300 of the canister is intentionally made devoid of (that is, necessarily does not include) a component configured to stir contents of the canister, according to the idea of the invention.

In operation, an external connector (such as a flexible tube), which is configured to receive the contents from the volume 116 (or 216, or the volume of the embodiment 300) through the tubular body or element 136 (or 236, or the corresponding tubular element of the embodiment 300), may be fluidly attached to the outlet port (132, 232, 332) and may be further complemented with at least a vacuum-forming means and/or a valve, if operationally required.

### Additional Considerations

In fabrication of embodiments of related art, an existing solid plastic container is modified by having a hole drilled in the side to install a separate, typically metallic hose barb outlet port. Such method of fabrication often results in biocompatibility issues and creates a lot of crevices and tight spaces for contaminants to hide, making cleaning less complete. When the canister of related art is structured to have the drainage of the contents thereof occur directly out the bottom (straight down), the canister requires a metal stand to lift it high enough off the table to install tubing for removing material. The discussed above embodiments include a one-piece, molded, single-component canister in which the base, the tubular element, and the outlet are all co-molded thereby naturally minimizing the room in which potential contaminants may be present, and making cleaning easier and more thorough. Our device will be able to be placed directly on a table and allow the user to install drainage tubing without the need for a stand.

Both the body of the canister and the body of the lid of the canister (with an exception of the O-ring) is preferably made entirely of one plastic material such as, for example, PSU Polysulfone, which is very chemical resistant, very strong, durable, long lasting and reusable.

In at least one specific embodiment, the article of manufacture may be, according to the idea of the invention, substantially completely clear and optically transparent to provide the user with the ability to observe substantially any portion or crevasse of the body of the article substantially in any orientation of the article to detect and remove any debris or foreign material during each cleaning between the uses. This includes the O-ring of the lid as well.

When the axis of the outlet is made tilted with respect to the axis of the canister, the corresponding a downward angle (about 9-10 degrees) that such axis of the outlet forms with a horizon helps the material flow out of the container, reducing any areas where the material could pool or collect.

The material and design are preferably rated for full vacuum (29.9 in Hg at sea level) even while used with aspirated material. Many, if not all, of the existing canister manufacturers out there do not rate their canisters to withstand max vacuum pressure when used in a liposuction procedure. This is believed to be because a chemical(s) or substance(s) present in the material removed from the patient interacts and structurally weakens the plastic material. There have been instances where these containers have failed and imploded during the procedure, causing aspirated material to be ejected everywhere. The solution has been to use proper materials (like our PSU) or to reduce the vacuum level, which slows the procedure. Some, but not all, existing containers use a thin, disposable plastic liner within a rigid plastic shell to collect the tissue and fat. This makes for an easy disposable, and may protect the weaker external rigid plastic from the debris as mentioned above. The proposed embodiments of the canister will not need this liner, and are designed to be reusable.

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, all refer to the same embodiment. It is to be understood that no portion of disclosure, taken on its own and in possible connection with a figure, is intended to provide a complete description of all features of the invention.

For the purposes of this disclosure and the appended claims, the use of the terms “substantially”, “approximately”, “about” and similar terms in reference to a descriptor of a value, element, property or characteristic at hand is intended to emphasize that the value, element, property, or characteristic referred to, while not necessarily being exactly as stated, would nevertheless be considered, for practical purposes, as stated by a person of skill in the art. These terms, as applied to a specified characteristic or quality descriptor means “mostly”, “mainly”, “considerably”, “by and large”, “essentially”, “to great or significant extent”, “largely but not necessarily wholly the same” such as to reasonably denote language of approximation and describe the specified characteristic or descriptor so that its scope would be understood by a person of ordinary skill in the art. In one specific case, the terms “approximately”, “substantially”, and “about”, when used in reference to a numerical value, represent a range of plus or minus 20% with respect to the specified value, more preferably plus or minus 10%, even more preferably plus or minus 5%, most preferably plus or minus 2% with respect to the specified value. As a non-limiting example, two values being “substantially equal” to one another implies that the difference between the two values may be within the range of +/−20% of the value itself, preferably within the +/−10% range of the value itself, more preferably within the range of +/−5% of the value itself, and even more preferably within the range of +/−2% or less of the value itself. The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

The use of these terms in describing a chosen characteristic or concept neither implies nor provides any basis for indefiniteness and for adding a numerical limitation to the specified characteristic or descriptor. As understood by a skilled artisan, the practical deviation of the exact value or characteristic of such value, element, or property from that stated falls and may vary within a numerical range defined by an experimental measurement error that is typical when using a measurement method accepted in the art for such purposes.

For the purposes of this disclosure and the appended claims, the expression of the type “element A and/or element B” is intended to be equivalent to and to have the same meaning as that of the expression “at least one of element A and element B”.

While the invention is described through the above-described exemplary embodiments, it will be understood by those of ordinary skill in the art that modifications to, and variations of, the illustrated embodiments may be made without departing from the inventive concepts disclosed herein. Disclosed aspects, or portions of these aspects, may be combined in ways not listed above. Accordingly, the invention should not be viewed as being limited to the disclosed embodiment(s).

1. An article of manufacture comprising:
a canister having a body and configured for use in autologous fat transfer and having a first axis, the body comprising:
a side wall;
a bottom having an inner surface inclined with respect to the first axis and a drain formed therein,
wherein the side wall and the bottom limit a volume of the canister,
wherein the volume of the canister includes a lower volume limited by the inner surface and a plane that is substantially normal to the first axis and that passes through an upper-most point of the inner surface,
wherein the volume of the canister includes an upper volume limited by said plane and an upper edge of the side wall;
an outlet port attached to the body; and
a tubular body having a second axis and configured to connect the drain and an outlet port,
wherein the second axis is tilted with respect to the first axis;
wherein the article is devoid of a component configured to stir contents of the canister.

2. An article of manufacturer according to claim 1, wherein at least one of the following conditions is satisfied:
the article further comprises an aspiration port in fluid communication with the upper volume,
and
wherein the article is devoid of a filter configured to filter out a portion of the contents of the canister upon removal thereof through the outlet port.

3. An article of manufacture according to claim 1, wherein a third axis of the outlet port is inclined with respect to the first axis and/or is substantially parallel to the second axis.

4. An article of manufacture according to claim 1, wherein the inner surface is a first substantially conical surface.

5. An article of manufacture according to claim 4,
wherein an outer surface of the side wall is a second substantially conical surface, and
wherein the first substantially conical surface is substantially centered at the first axis.

6. An article of manufacture according to claim 1,
wherein the body further comprises a base that is circumferential to the first axis and that engages the side wall below the bottom,
wherein the outlet port is attached to the base at a level that is farther away from the upper edge than a level of the drain.

7. An article of manufacture according to claim 6, wherein:
the base includes an upper base portion merging with the side wall and a pedestal portion that merges with the upper base portion and that has a diameter exceeding a diameter of the upper base portion,
and/or
a first thickness of a wall of the base and a second thickness of the side wall are substantially equal to one another and remain substantially the same at any location at the article of manufacture.

8. An article of manufacture according to claim 7, wherein an upper surface of the pedestal is dimensioned substantially as an annulus and/or said upper surface of the pedestal does not lie in a plane.

9. An article of manufacture according to claim 6,
wherein an outer surface of the base contains an opening therethrough or a notch dimensioned to substantially fully accommodate the outlet port therein to prevent the outlet port from protruding outside the outer surface while not obstructing the outlet port from being accessed and/or cooperated with the tubular body.

10. An article of manufacture according to claim 1, wherein each portion of the article is configured to be substantially optically transparent.

11. An article of manufacture comprising:
a canister configured for use in autologous fat transfer and having a body with a first axis,
the body comprising:
a side wall;
a bottom having an inner surface inclined with respect to the first;
wherein the side wall and the bottom limit a volume of the canister;
a top removably engaged with the side wall to create a first seal with the side wall;
a base having a base wall with a base height, the base wall being circumferential to the first axis and engaging the side wall below the bottom;
an outlet port attached directly to the base in fluid communication with the volume; and
a tubular body configured below the bottom to fluidly connect the inner surface and the outlet port, the tubular body having a second axis that is tilted with respect to the first axis.

12. An article of manufacture according to claim 11,
wherein a lower edge of the base wall defines a base aperture leading to a base volume limited by an outer surface of the bottom and the base wall, and
wherein the tubular body is substantially completely disposed within the base volume.

13. An article of manufacture according to claim 11, wherein the first and second axes intersect.

14. An article of manufacture according to claim 11, wherein the volume of the canister includes:
a lower volume limited by the inner surface and a plane that is substantially normal to the first axis and that passes through an upper-most point of the inner surface, and
an upper volume limited by said plane and an upper edge of the side wall.

15. An article of manufacture according to claim 11, wherein the outlet port is directly attached to the base at a level that is farther away from the upper edge than a level of the drain.

16. An article of manufacture according to claim 15, wherein at least one of the following conditions is satisfied:
(i) a third axis of the outlet port is inclined with respect to the first axis and/or is substantially parallel to the second axis, and
(ii) the article is devoid of a component configured to stir contents of the canister.

17. An article of manufacture according to claim 11, wherein an outer surface of the base contains an opening therethrough or a notch dimensioned to substantially fully accommodate the outlet port therein to prevent the outlet port from protruding outside the outer surface while, at the same time, not obstructing the outlet port from being accessed and/or cooperated with the tubular body.

18. An article of manufacture according to claim 11, wherein each portion of the article is configured to be optically transparent.

19. A method comprising:
with the use of the article of manufacture according to claim 11:
maintain a lipoaspirate matter contained within the volume of the canister without agitation to have said lipoaspirate matter separate into an unwanted fluid component in contact with the inner surface of the bottom and a fat component on top of the unwanted fluid component;
extracting the unwanted fluid component through the outlet port along the second axis.
